(19) United States
(12) Patent Application Publication
(10) Pub. No.: US 2023/0310079 A1
(43) Pub. Date: Oct. 5, 2023

(54) PROVIDING A RESULT DATA SET

(71) Applicant: Siemens Healthcare GmbH, Erlangen (DE)

(72) Inventor: Thomas Redel, Poxdorf (DE)

(21) Appl. No.: 18/124,724

(22) Filed: Mar. 22, 2023

(30) Foreign Application Priority Data
Mar. 31, 2022 (DE) 10 2022 203 162.8

(51) Int. Cl. A61B 34/10; A61B 34/20

(52) U.S. Cl. CPC A61B 34/10; A61B 34/20; A61B 2034/2068; A61B 2034/2055

(57) ABSTRACT

A method for providing a result data set includes providing a first planning image that maps a first physiological phase of a motion of an object under examination, wherein the first planning image contains planning information about a planned positioning of a medical object in the object under examination. The method further includes acquiring a first monitoring image that maps the first physiological phase of the motion of the object under examination and the medical object arranged in the object under examination. The method further includes providing the result data set: (a) based on the first planning image and positioning information, which is determined by identification of a mapping of the medical object in the first monitoring image, or (b) based on the planning information and the first monitoring image.

The present patent document claims the benefit of German Patent Application No. 10 2022 203 162.8, filed Mar. 31, 2022, which is hereby incorporated by reference in its entirety.

### TECHNICAL FIELD

The present disclosure relates to a method for providing a result data set, to a medical imaging device and to a computer program product.

### BACKGROUND

In the case of coronary heart disease, plaque accumulates inside the arteries and causes a constriction of the arterial vessel walls and a reduction in the diameter of the vessel. This may result in a stenosis. One possibility for treating a stenosis is to place a stent in the region to be treated of the vessel. The current guidelines on planning such a treatment recommend the “normal-to-normal” or “healthy-to-healthy” strategy. The basic idea is that not only the stenotic region should be covered by the stent, but also adjacent pathological regions of the vessel that contain fibrotic plaque structures. On the other hand, the stent may be selected to be as short as possible, because longer stents may increase the risk of a new stenosis inside the stent. The stent is frequently positioned under fluoroscopic imaging monitoring. Before the stent is positioned, an angiography scene is frequently acquired by a C-arm X-ray device, while a radiopaque contrast agent is injected into the coronary artery. The angiography scene may be acquired with an identical positioning of the C-arm X-ray device as for the subsequent uncontrasted fluoroscopic images.

In the fluoroscopic images, the manual positioning of the stent may be monitored using radiopaque markers which are attached to the stent and are readily identifiable in the fluoroscopic images. A disadvantage of this is that the coronary arteries, in particular the contrasted coronary arteries, planning information and/or the stenosis cannot be observed in the fluoroscopic images.

To assist medical operating personnel in positioning a medical object, (e.g., a stent), in an object under examination, an image may be selected from a pre-captured angiography scene, which includes the coronary artery, the stenosis, and planning information, for example, from intravascular imaging or pre-interventional imaging by computed tomography (CT) or magnetic resonance tomography (MRT). This image may then be displayed side-by-side with a fluoroscopic real-time image, in order to be able to provide the medical operating personnel with at least rough information about the position of the stent in respect of the stenosis. A disadvantage of this is that the medical operating personnel have to compare two graphical presentations displayed side-by-side, wherein a movement of the stent is displayed in the real-time image and both the graphical presentations correlate with one another only at one point in a cardiac phase.

Dynamic coronary road mapping techniques are a further possibility, wherein multiple mask images are generated from the images of an angiography scene and are linked in each case with one point in a cardiac phase, for example, by an EKG. During the acquisition of the fluoroscopic images, an EKG signal may be acquired and the associated mask image superimposed. Additionally, in this case, a respiratory motion of the object under examination may be compensated additively. This approach is based on EKG-based matching, wherein deviations may occur because the cardiac motion is not fully reproducible. In particular, after a catheter has been introduced into the coronary artery an irregular heartbeat may occur which deviates compared to the heartbeat mapped in the angiography scene. Furthermore, in this case the medical operating personnel lack the planning information needed to position the stent.

### SUMMARY AND DESCRIPTION

The object of the disclosure is hence to enable improved imaging-based support when positioning a medical object in an object under examination.

The scope of the present disclosure is defined solely by the appended claims and is not affected to any degree by the statements within this summary. The present embodiments may obviate one or more of the drawbacks or limitations in the related art.

A first aspect of the disclosure relates to a method for providing a result data set. In this case, the method includes providing a first planning image, which maps a first physiological phase of a motion of an object under examination. In this example, the first planning image contains planning information about a planned positioning of a medical object in the object under examination. The method further includes acquiring a first monitoring image, which maps the first physiological phase of the motion of the object under examination and the medical object arranged in the object under examination. The method further includes providing the result data set. In accordance with a first variant, the result data set is provided based on the first planning image and the positioning information, which is determined by identification of a mapping of the medical object in the first monitoring image. In accordance with a second variant, the result data set is provided based on the planning information and the first monitoring image.

The terms “first planning image,” “first monitoring image,” and “first physiological phase” do not in this case imply a sequence, in particular not a time of acquisition.

The provision of the first planning image may include a receipt and/or acquisition of the first planning image. The receipt of the first planning image may include a capture and/or readout of a computer-readable data store and/or a receipt from a data storage unit, for example, a database. For example, the first planning image may be provided by a provision unit of a medical imaging device for the acquisition of the first planning image. Alternatively, or additionally, the first planning image may be acquired by a medical imaging device. The medical imaging device for the acquisition of the first planning image may be configured as a magnetic resonance tomography installation (MRT installation) and/or a computed tomography installation (CT installation) and/or a medical X-ray device and/or a positron emission tomography installation (PET installation) and/or an ultrasound device, in particular an intravascular ultrasound device, and/or an imaging device for optical coherence tomography (OCT).

The object under examination may be a male and/or female human and/or animal patient, and/or a phantom under examination, in particular a vascular phantom. The first planning image may map the first physiological phase of the motion of the object under examination two-dimensionally (2D) and/or three-dimensionally (3D) on a spatially resolved basis. Further, the first planning image may map the first physiological phase of the motion of the object under examination on a time-resolved basis. The motion of the object under examination may include a motion of at least one part of the object under examination, in particular of an anatomical object, for example, an organ and/or tissue, and/or of a body part, for example, a cardiac motion and/or a respiratory motion. In particular, the motion of the object under examination may be substantially periodic. The motion of the object under examination may contain multiple temporal sections, in particular periodically recurring temporal sections, for example, as a function of a speed of motion and/or direction of motion and/or amplitude of motion. In this case, the first physiological phase of the motion may include a temporal section of the motion. The first planning image may advantageously map the object under examination in a first temporal phase, in particular pre-procedurally.

The first monitoring image may be acquired by the medical imaging device for the acquisition of the first planning image or by a further medical imaging device. The further medical imaging device may be configured as an MRT installation and/or a CT installation and/or a medical X-ray device and/or a PET installation and/or an ultrasound device. The first monitoring image may map the first physiological phase of the motion of the object under examination and the medical object arranged in the object under examination in 2D and/or 3D on a spatially resolved basis. For this, the acquisition of the first monitoring image may take place on a triggered basis, for example, using a physiological signal which is provided by a sensor, for example, an EKG sensor, for the capture of the motion of the object under examination. The medical object may be configured as a surgical and/or diagnostic instrument, for example, as an endoscope and/or laparoscope and/or catheter and/or guidewire, and/or as an implant, for example, as a stent and/or microwire. The first monitoring image may advantageously map the object under examination in a second temporal phase, in particular intra-procedurally. In this case, the medical object may be arranged within the second temporal phase, in particular intra-procedurally, in the object under examination, in particular a hollow organ of the object under examination.

The first planning image may advantageously contain planning information about a planned positioning, in particular a planned spatial position and/or orientation and/or pose, of the medical object in the object under examination. The planning information may advantageously be specified manually and/or semi-automatically and/or fully automatically using the first planning image, for example, by annotation of the first planning image using a user input.

The provision of the result data set may include storage on a computer-readable storage medium and/or display on a presentation unit and/or transfer to a provision unit. In particular, a graphical presentation of the result data set may be displayed by the presentation unit.

In accordance with a first variant, the result data set may be provided on the basis of the first planning image and the positioning information. In this case, the mapping of the medical object may be identified by identification, in particular segmentation, of image points, in particular pixels and/or voxels, of the first monitoring image, the image points mapping the medical object. The identification, in particular segmentation, of the image points in the first monitoring image may include a comparison of image values of the image points with a threshold value and/or use of an algorithm for pattern or marker recognition. The medical object may advantageously contain at least one marker structure mapped in the first monitoring image.

Advantageously, using the mapping of the medical object identified in the first monitoring image, the positioning information containing information about the spatial position and/or orientation and/or pose of the medical object may be determined in the first physiological phase of the motion of the object under examination.

In accordance with a second variant, the result data set may be provided on the basis of the planning information and the first monitoring image.

The provision of the result data set may include at least a partial, in particular complete, overlapping and/or merging of the first planning image with the positioning information or of the first monitoring image with the planning information.

As a result, improved imaging-based support for the medical operating personnel may advantageously be enabled during the positioning of the medical object in the object under examination.

In a further advantageous embodiment, at least one further monitoring image may be acquired, which maps at least one further physiological phase of the motion of the object under examination and the medical object arranged in the object under examination. In this case, positioning information about the first and the at least one further monitoring image may be determined by identification in each case of a mapping of the medical object in the first and the at least one further monitoring image. Further, a transformation rule between the positioning information of the first monitoring image and the positioning information of the at least one further monitoring image may be determined. Further, the result data set may be provided on the basis of the transformation rule.

The at least one further monitoring image may in particular contain all features and properties of the first monitoring image. The at least one further monitoring image may advantageously be acquired by the medical imaging device for the acquisition of the first monitoring image. In particular, multiple further monitoring images may be acquired, which map multiple further physiological phases of the motion of the object under examination and the medical object arranged in the object under examination. In this case, one of the multiple further monitoring images may in each case map one of the multiple physiological phases of the motion of the object under examination in 2D or 3D on a spatially resolved basis.

In this case, the at least one further physiological phase of the motion may include a further temporal section of the motion. In particular, the first and the at least one further physiological phase, in particular the multiple further physiological phases, of the motion in a direct temporal sequence may form the motion of the object under examination.

The determination of the positioning information about the at least one further monitoring image, in particular about the multiple further monitoring images, may in particular take place analogously to the determination of the positioning information about the first monitoring image. In particular, the identification of the mapping of the medical object in the at least one further monitoring image, in particular in the multiple further monitoring images, may take place analogously to the identification of the mapping of the medical object in the first monitoring image.

The transformation rule may specify an, in particular rigid or non-rigid, translation and/or rotation and/or scaling and/or deformation, by which the positioning information of the first monitoring image and the positioning information of the at least one further monitoring image may be mapped spatially to one another. The determination of the transformation rule may include a minimization of a deviation between the positioning information of the first monitoring image and the positioning information of the at least one further monitoring image. In particular, the positioning information of the first monitoring image and the positioning information of the at least one further monitoring image may be registered with one another using the transformation rule. In this case the transformation rule may be configured to be applied to the positioning information and/or the monitoring images. In particular, the provision of the result data set may include an application of the transformation rule to the positioning information or the monitoring images. As a result, a deviation between the positioning information and/or the monitoring images caused by the motion of the object under examination may advantageously be compensated.

In a further advantageous embodiment, the result data set may be provided on the basis of the first planning image and adjusted positioning information, which is provided by applying the transformation rule to the positioning information.

The provision of the result data set may include an at least partial, in particular complete, overlapping and/or merging of the first planning image with the adjusted positioning information. As a result, improved imaging-based support may be enabled when positioning a medical object in the object under examination. In particular, the deviation between the positioning information, in particular in respect of the first physiological phase of the motion, caused by the motion of the object under examination, may advantageously be compensated. Furthermore, the deviation between the positioning information and the first planning image caused by the motion of the object under examination may advantageously be minimized.

In a further advantageous embodiment, the result data set may be provided on the basis of the planning information and adjusted monitoring images, which are provided by applying the transformation rule to the first and the at least one further monitoring image.

In this case, the multiple monitoring images may be registered with one another in respect of the positioning information of the first monitoring image by applying the transformation rule to the monitoring images. As a result, improved imaging-based support may be enabled when positioning the medical object in the object under examination. In particular, the deviation between the monitoring images, in particular in respect of the first physiological phase of the motion, caused by the motion of the object under examination, may advantageously be compensated. Furthermore, the deviation between the monitoring images and the planning information caused by the motion of the object under examination may advantageously be minimized.

In a further advantageous embodiment, the provision of the result data set may include a summing, in particular addition, of the adjusted monitoring images.

The monitoring images, in particular the first and the at least one further monitoring image, may advantageously be acquired at a higher image acquisition rate, in particular compared to the first planning image. Thanks to the summing, in particular addition, of the adjusted monitoring images an image quality, in particular a signal-to-noise ratio and/or a contrast-to-noise ratio, may be improved compared to the individual adjusted monitoring images. Further, as a result the image quality of the result data set may advantageously be improved.

In a further advantageous embodiment, the determination of the transformation rule may include a determination of a field of motion between the mappings of the medical object in the first and the at least one further monitoring image. In this case, the transformation rule may be determined on the basis of the field of motion.

The field of motion may contain an, in particular 2D or 3D, spatially and temporally resolved representation, in particular a model, of the motion of the medical object mapped in the first and the at least one further monitoring image. In this case, the field of motion may represent, (e.g., may model), on a temporally and spatially resolved basis, a change in the positioning of the medical object during the motion of the object under examination mapped in the first and the at least one further monitoring image. In particular, the field of motion may represent a change in the positioning information of the first and the at least one further monitoring image. The field of motion may contain a vector field or tensor field representing the motion of the medical object. In this case, the motion of the medical object represented, in particular modeled, in the field of motion may include a translation and/or rotation and/or deformation of at least one part of the medical object. Further, the transformation rule may be determined on the basis of the field of motion, in particular on the basis of the motion of the medical object represented, in particular modeled, in the field of motion, such that the positioning information of the first monitoring image and the positioning information of the at least one further monitoring image may be spatially mapped to one another by applying the transformation rule to the positioning information or by applying the transformation rule to the monitoring images. In particular, by applying the transformation rule to the positioning information a deviation between the positioning information of the first and the at least one further monitoring image may be minimized. Alternatively, by applying the transformation rule to the first and the at least one further monitoring image a deviation between the first and the at least one further monitoring image may be minimized.

By the proposed embodiment, the adjusted positioning information, or the adjusted monitoring images may be provided particularly precisely. As a result, an improved provision of the result data set may be enabled.

In a further advantageous embodiment, the first monitoring image may be acquired by a physiological signal which maps the physiological phases of the motion, in particular at least the first physiological phase of the motion.

The acquisition of the first monitoring image may advantageously be triggered by the physiological signal. The physiological signal may advantageously map the physiological phases of the motion, in particular the first physiological phase of the motion, in particular on a time-resolved basis. The physiological signal may advantageously be provided by a sensor, for example, an EKG sensor, for the capture of the motion of the object under examination. In this case, the acquisition of the first monitoring image may be triggered by the physiological signal, such that the first monitoring image maps the first physiological phase of the motion. The first physiological phase of the motion may in this case be specified as a function of the first planning image, in particular as a function of the first physiological phase of the motion mapped in the first planning image.

The proposed embodiment may enable an acquisition of the first monitoring image which is particularly efficient in terms of time and/or dose.

In a further advantageous embodiment, the motion of the object under examination may include a cardiac motion. In this case, the first physiological phase may be specified as a diastolic phase of the cardiac motion.

The cardiac motion may include a periodic sequence of contractions and relaxations of the heart of the object under examination. In this case, the multiple physiological phases of the motion, in particular of the cardiac motion, of the object under examination may include a systolic phase and a diastolic phase. The systolic phase may describe a period of the contraction of the heart of the object under examination. Further, the diastolic phase may describe a period of the relaxation of the heart of the object under examination. The first physiological phase may advantageously be specified as the diastolic phase of the cardiac motion, in particular as the period of the relaxation of the heart of the object under examination.

Because the cardiac motion is minimal during the diastolic phase, an improved provision of the result data set may be enabled. In particular, deviations between the first planning image and the positioning information, in particular the adjusted positioning information, or between the planning information and the first monitoring image, in particular the adjusted monitoring images, may advantageously be minimized.

In a further advantageous embodiment, the medical object may contain at least one marker structure. In this case, the determination of the positioning information may take place by identification of a mapping of the at least one marker structure.

The at least one marker structure may contain an imaging-visible, in particular contrast-enhancing and/or radiopaque, structure, for example, a fiducial marker. The at least one marker structure may advantageously be integrated into the medical object or arranged on, in particular fastened to, the medical object.

The determination of the positioning information may include identification of a mapping of the at least one marker structure in the first monitoring image. If at least one further monitoring image is acquired, the determination of the positioning information may furthermore include identification of a mapping of the at least one marker structure in the at least one further monitoring image. In this case, the mapping of the at least one marker structure may be identified by identification, in particular segmentation, of image points, in particular pixels and/or voxels, of the respective monitoring image, the image points mapping the at least one marker structure. The identification, in particular segmentation, of the image points in the respective monitoring image may include a comparison of image values of the image points with a threshold value and/or an application of an algorithm for pattern or marker recognition. The threshold value may be specified as a function of a material parameter of the at least one marker structure. Advantageously, using the mapping of the at least one marker structure identified in the first monitoring image, positioning information containing information about the spatial position and/or orientation and/or pose of the at least one marker structure, in particular of the medical object, may be determined in the first physiological phase of the motion of the object under examination. In particular, using the mapping of the at least one marker structure identified in the first and the at least one further monitoring image, positioning information containing information about the spatial position and/or orientation and/or pose of the at least one marker structure, in particular of the medical object, may respectively be determined in the first and the at least one further physiological phase of the motion of the object under examination.

The proposed embodiment may enable an improved, in particular more precise, determination of the positioning information.

In a further advantageous embodiment, the medical object may contain a spatial arrangement of multiple marker structures. In this case, the planning information may contain planning positioning in the object under examination for the spatial arrangement of the multiple marker structures. Furthermore, the determination of the positioning information may include identification of a mapping of the spatial arrangement of the multiple marker structures.

The medical object may advantageously contain a spatial arrangement, in particular an arrangement defined in respect of the medical object, of the multiple marker structures. In this case the multiple marker structures may be configured to be identical or different. Further, the spatial arrangement of the multiple marker structures may describe a defined positioning, in particular relative positioning, of the multiple marker structures in respect of the medical object and/or a defined positioning, in particular relative positioning, of the multiple marker structures to one another. The multiple marker structures may advantageously be arranged as stationary in respect of the medical object, in particular fastened to the medical object. Furthermore, the multiple marker structures may be arranged, in particular fastened, on the medical object in a defined arrangement to one another or dynamically to one another. In the case of a dynamic arrangement of the multiple marker structures on the medical object a pose, for example, a curvature, of the medical object may be mapped by the mapping of the multiple marker structures, in particular in the first monitoring image.

The planning information may advantageously contain, in particular specify, the planning positioning, in particular a planned spatial position and/or orientation and/or pose, for the spatial arrangement of the multiple marker structures in the object under examination.

Further, the determination of the positioning information may include identification of a mapping of the spatial arrangement of the multiple marker structures in the first monitoring image. If at least one further monitoring image is acquired, the determination of the positioning information may furthermore include identification of a mapping of the spatial arrangement of the multiple marker structures in the at least one further monitoring image. In this case, the respective mapping of the spatial arrangement of the multiple marker structures may be identified by identification, in particular segmentation, of image points, in particular pixels and/or voxels, of the respective monitoring image, the image points mapping the spatial arrangement of the multiple marker structures. The identification, in particular segmentation, of the image points in the respective monitoring image may include a comparison of image values of the image points with a threshold value and/or an application of an algorithm for pattern or marker recognition. The threshold value may be specified as a function of a material parameter of the multiple marker structures. Advantageously, using the mappings of the spatial arrangement of the multiple marker structures identified in the first monitoring image, positioning information containing information about the spatial position and/or orientation and/or pose of the spatial arrangement of the multiple marker structures, in particular of the medical object, may be determined in the first physiological phase of the motion of the object under examination. In particular, using the mappings of the spatial arrangement of the multiple marker structures identified in the first and the at least one further monitoring image, in each case positioning information containing information about the spatial position and/or orientation and/or pose of the spatial arrangement of the multiple marker structures, in particular of the medical object, may be determined in the respectively mapped physiological phase of the motion of the object under examination.

The proposed embodiment may enable an improved, in particular more precise, determination of the positioning information, in particular in respect of an orientation and/or pose of the medical object in 3D.

In a further advantageous embodiment, the acquisition of the first monitoring image may take place with an acquisition geometry which corresponds to an acquisition geometry of the first planning image in respect of the object under examination.

The first monitoring image, in particular also the at least one further monitoring image, and the first planning image may advantageously be acquired with a substantially identical acquisition geometry of the respective medical imaging device, in particular by the same medical imaging device. The acquisition geometry may include a spatial arrangement of the medical imaging device in respect of the object under examination, in particular an angulation, and/or a mapping geometry of the medical imaging device, for example, a mapping window (field-of-view, FOV) and/or an aperture angle, and/or an acquisition parameter of the medical imaging device. Thanks to the acquisition of the first monitoring image with a substantially identical acquisition geometry in respect of the object under examination as for the acquisition of the first planning image, a motion-independent deviation between the first planning image and the first monitoring image, in particular between the planning information and the positioning information, may advantageously be minimized. In contrast to deviations in the case of acquisition geometry, a deviation of operating parameters during the acquisition of the first planning image and the first monitoring image, for example, a motion of a patient support device and/or a zoom and/or a distance between source and detector (source image receptor distance, SID) may be corrected using information about these operating parameters.

In a further advantageous embodiment, the first planning image may map a contrasted hollow organ of the object under examination. In this case, the medical object may be arranged at least partially in the hollow organ during the acquisition of the first monitoring image, in particular also during the acquisition of the at least one further monitoring image.

The hollow organ may include a section of a vessel, in particular an artery and/or vein. In this case, the first planning image may advantageously map a contrast agent arranged in the hollow organ, for example, a radiopaque contrast agent. A hollow organ filled at least partially with contrast agent may be referred to a contrasted hollow organ. Furthermore, the first monitoring image may map the medical object, which within the second temporal phase is arranged at least partially in the hollow organ.

In particular, when the result data set is provided on the basis of the first planning image and the positioning information, in particular the adjusted positioning information, the result data set may advantageously contain the mapping of the contrast agent arranged in the hollow organ and the positioning information, in particular the adjusted positioning information. As a result, particularly intuitive support for the medical operating personnel may be enabled during the positioning of the medical object in the hollow organ.

In a further advantageous embodiment, the acquiring of the first monitoring image and the providing of the result data set may be executed repeatedly.

The acquiring and the providing may be executed repeatedly for a predetermined number of maximum repetitions and/or until a termination condition occurs. In particular, the repeated execution may be actuated, in particular triggered, by a motion of the medical object. The actuation of the repeated execution may take place manually or automatically. For a manual actuation of the repeated execution of the acquiring and the providing, a further user input may be captured by an input unit, for example, a keyboard and/or a key and/or a button and/or a lever and/or a pedal. Alternatively, the repeated execution of the acquiring and the providing may be actuated automatically, in particular during an identification of a motion of the medical object. For this, an object signal may be received by a capture unit for the capture of a motion of the medical object, wherein the object signal includes information about a direction of motion and/or speed of motion and/or amplitude of motion of the medical object. The capture unit may be integrated into the medical object, arranged on a target point for the motion of the medical object or arranged at a distance from the medical object. The capture unit may include a mechanical and/or electromagnetic, in particular optical, and/or acoustic and/or gyroscopic sensor, which is configured for the capture of the motion of the medical object.

It is advantageously further possible for the physiological signal to be received, which maps the physiological phases of the motion of the object under examination. The physiological signal may be received by the sensor for the capture of the motion of the object under examination, for example, a respiratory sensor and/or an EKG sensor. The object signal may advantageously be motion-corrected by the physiological signal. As a result, an actual motion of the medical object in respect of the object under examination may be differentiated from a motion of the medical object following on from the motion of the object under examination.

Thanks to the proposed embodiment, more reliable monitoring of the positioning of the medical object may be enabled.

In a further advantageous embodiment, the first planning image and the first monitoring image may be acquired by the same or different medical imaging devices, in particular imaging modalities.

If the first planning image and the first monitoring image are acquired by the same medical imaging device, imaging-induced deviations between the first planning image and the first monitoring image may advantageously be minimized. As a result, the transformation rule may be particularly reliably determined. In particular, the at least one further monitoring image may also be acquired by the same medical imaging device as the first planning image and the first monitoring image.

Alternatively, the first planning image and the first monitoring image may be acquired by different, in particular dedicated, medical imaging devices. In particular, the at least one further monitoring image may be acquired by the same medical imaging device as the first monitoring image and by a medical imaging device different from the medical imaging device for the acquisition of the first planning image. As a result, an improved acquisition, in particular optimized to the respective imaging target, of the first planning image and the first monitoring image may be enabled. In this case the medical imaging device for the acquisition of the first planning image may in particular map anatomical objects, for example, an organ, in particular the hollow organ, and/or tissue, with a high image quality and/or a high level of detail. Further, the medical imaging device for the acquisition of the first monitoring image may map the medical object, in particular the at least one marker structure, at a high image acquisition rate.

In a further advantageous embodiment, a motion correction for the compensation of a further motion, in particular a respiratory motion, of the object under examination may be applied to the first planning image and the first monitoring image.

During and/or between the acquisition of the first planning image and/or the acquisition of the first monitoring image the object under examination may have a further motion, for example, a respiratory motion. This further motion may cause a divergent mapping of the object under examination in the first physiological phase of the motion of the object under examination in the first planning image and the first monitoring image.

The motion correction may include an application of a further transformation rule to the first planning image and/or the first monitoring image. By applying the further transformation rule to the first planning image and/or the first monitoring image, the first planning image and the first monitoring image may be registered. The further transformation rule may specify an, in particular rigid or non-rigid, translation and/or rotation and/or scaling and/or deformation, thanks to which a deviation between the first planning image and the first monitoring image, caused by the further motion, may be minimized. The motion correction, in particular the further transformation rule, may be determined using a further physiological signal, which maps the further motion, and/or may be image-based, in particular using a shared mapping in the first planning image and the first monitoring image. The further physiological signal may be provided by a further sensor for the capture of the further motion, for example, a respiratory sensor.

As a result, the further motion, in particular the respiratory motion, of the object under examination may advantageously be compensated. In particular, the deviation between the first planning image and the first monitoring image induced by the further motion may be minimized.

In a further advantageous embodiment, the first planning image and the first monitoring image may map a predefined section of the medical object. In this case, the motion correction may be based on the shared mapping of the predefined section in the first planning image and the first monitoring image.

The motion correction, in particular the further transformation rule, may advantageously be determined on the basis of the shared mapping of the predefined section. In this case, the determination of the motion correction may include identification, in particular segmentation, of the shared mapping of the predefined section in the first planning image and the first monitoring image. The predefined section of the medical object may be a distal section, for example, a tip of a catheter. The identification of the shared mapping of the predefined section may include a comparison of image values of the image points of the first planning image and the first monitoring image within each case a threshold value. Alternatively, the shared mapping of the predefined section may be identified using geometric features, for example, a contour.

The determination of the motion correction, in particular of the further transformation rule, may include a minimization of a deviation between the shared mapping of the predefined section of the medical object. As a result, the further motion, in particular the respiratory motion, of the object under examination may be compensated particularly precisely.

In a further advantageous embodiment, multiple planning images may be received which map multiple physiological phases of the motion of the object under examination. Further, multiple monitoring images may be acquired which map at least one part of the multiple physiological phases of the motion of the object under examination and the medical object arranged in the object under examination. In this case, the first planning image may be identified from the multiple planning images and the first monitoring image from the multiple monitoring images, such that the first planning image and the first monitoring image map the first physiological phase as a coincident physiological phase of the motion.

The coincident physiological phase may be specified, for example, with a user input. Alternatively, the coincident physiological phase may be determined, in particular semi-automatically or fully automatically, for example, using a comparison of the physiological phases of the motion, which are mapped in the multiple planning images and the multiple monitoring images. The coincident physiological phase of the motion may be identified using a coincident speed of motion and/or direction of motion and/or amplitude of motion of the respectively mapped motion of the object under examination. Thus the first planning image and the first monitoring image may map the object under examination in a coincident motion status.

A second aspect of the disclosure relates to a medical imaging device configured for the execution of a proposed method for providing a result data set.

The advantages of the proposed medical imaging device substantially correspond to the advantages of the proposed method for providing a result data set. Features, advantages, or alternative forms of embodiment mentioned here may likewise also be transferred to the other claimed subject matters and vice versa.

The medical imaging device may contain an imaging unit and a provision unit. In this case, the imaging unit may be configured for the acquisition of the first planning image and the first monitoring image. Further, the provision unit may be configured for the execution of the proposed method for providing a result data set.

A third aspect of the disclosure relates to a computer program product with a computer program that may be loaded directly into a memory of a provision unit, with program sections in order to execute all acts of a proposed method for providing a result data set, when the program sections are executed by the provision unit.

The disclosure may further relate to a computer-readable storage medium, on which program acts are stored that may be read and executed by a provision unit, in order to execute all acts of the method for providing a result data set, when the program sections are executed by the provision unit.

A software-based implementation has the advantage that provision units already previously in use may easily be retrofitted by a software update in order to work in the inventive manner. Such a computer program product may where appropriate include, besides the computer program, additional elements such as documentation and/or additional components, as well as hardware components, such as hardware keys (dongles, etc.) for the use of the software.

### BRIEF DESCRIPTION OF THE DRAWINGS

Exemplary embodiments of the disclosure are represented in the drawings and are described in greater detail below. The same reference characters are used in different figures for identical features. In the drawings:

FIGS. 1, 3, and 4 depict schematic presentations of different advantageous forms of embodiment of a method for providing a result data set.

FIG. 2 depicts a schematic presentation of an example of a first planning and monitoring image and of a result data set.

FIG. 5 depicts a schematic presentation of an example of a medical C-arm X-ray device.

### DETAILED DESCRIPTION

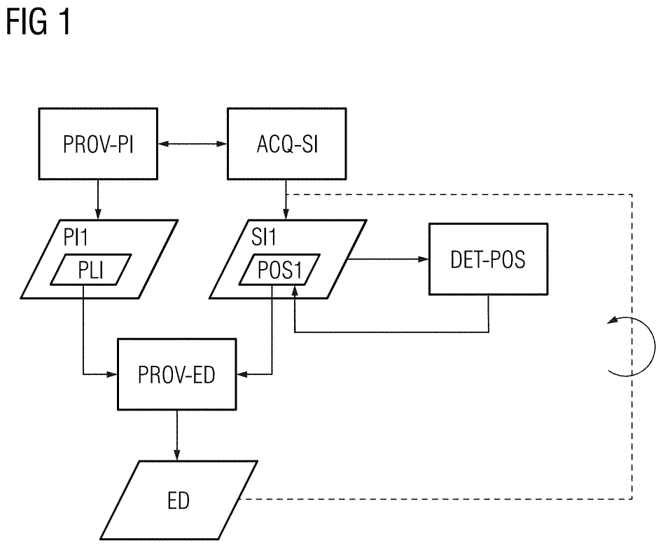

FIG. 1 schematically shows an advantageous embodiment of a proposed method for the provision PROV-ED of a result data set ED. In act a), a first planning image PI1 may be provided PROV-PI1, which maps a first physiological phase of a motion of an object under examination. Further, the first planning image PI1 may contain planning information PLI about a planned positioning of a medical object in the object under examination. In act b), a first monitoring image SI1 may be acquired ACQ-SI1, which maps the first physiological phase of the motion of the object under examination and the medical object arranged in the object under examination. The first planning image PI1 and the first monitoring image SI1 may advantageously be acquired by the same or by different medical imaging devices, in particular imaging modalities. In act c), the result data set ED may be provided PROV-ED on the basis of the first planning image PI1 and the positioning information POS1, the positioning information being determined DET-POS by identification of a mapping of the medical object in the first monitoring image SI1.

The motion of the object under examination may include a cardiac motion, wherein the first physiological phase is specified as a diastolic phase of the cardiac motion.

The medical object may advantageously contain at least one marker structure. In this case, the determination DET-POS of the positioning information POS1 may take place by identification of a mapping of the at least one marker structure. In particular, the medical object may contain a spatial arrangement of multiple marker structures. In this case, the planning information PLI may contain a planning positioning in the object under examination for the spatial arrangement of the multiple marker structures. Furthermore, the determination DET-POS of the positioning information POS1 may include identification of a mapping of the spatial arrangement of the multiple marker structures.

The acquisition ACQ-SI of the first monitoring image SI1 may advantageously take place with an acquisition geometry that corresponds to an acquisition geometry of the first planning image PI1 in respect of the object under examination.

The first planning image PI1 may advantageously map a contrasted hollow organ of the object under examination. In this case, the first monitoring image SI1 may map the medical object arranged at least partially in the hollow organ within the second temporal phase.

Furthermore, acts b) and c) may be executed repeatedly.

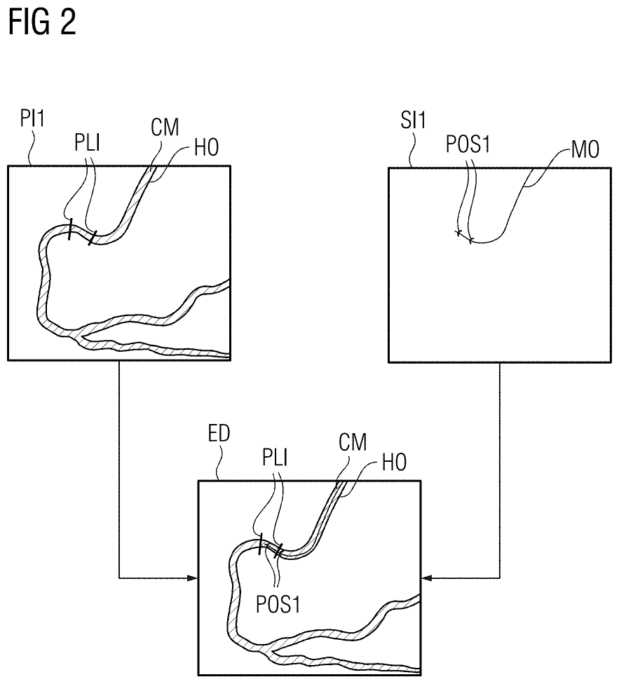

FIG. 2 schematically shows a first planning image PI1, a first monitoring image SI1, and a result data set ED. The first planning image PI1 may contain a mapping of a contrasted hollow organ HO. In this case, a contrast agent CM arranged in the hollow organ HO may be mapped in the first planning image PI1. Further, the first planning image PI1 may contain the planning information PLI, for example, a graphical specification for the positioning of the medical object MO. The first monitoring image SI1 may contain a mapping of the medical object MO and at least one marker structure arranged on the medical object MO. In this case, the positioning information POS1 may be identified ID-POS by identification of the mapping of the at least one marker structure in the first monitoring image SI1.

The result data set ED may advantageously be provided PROV-ED on the basis of the first planning image PI1 and the positioning information POS1, for example, by an overlapping of the first planning image PI1 with the positioning information POS1.

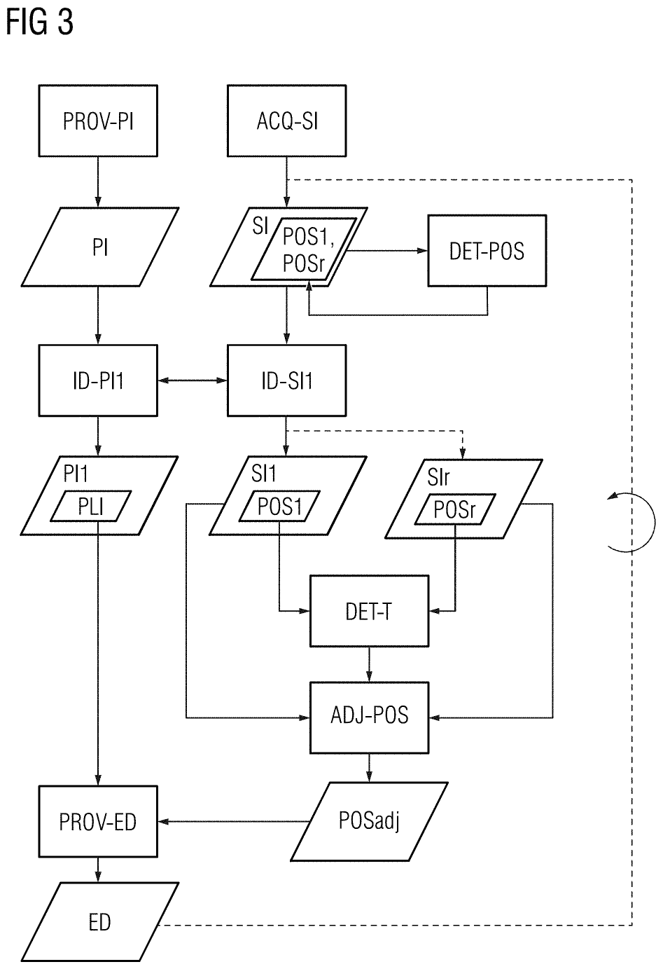

FIG. 3 shows a schematic presentation of a further advantageous embodiment of a proposed method for the provision PROV-ED of a result data set ED.

In this case, in act a), multiple planning images PI may be provided PROV-PI, which map multiple physiological phases of the motion of the object under examination. Further, in act b), multiple monitoring images SI may be acquired ACQ-SI, which map at least one part of the multiple physiological phases of the motion of the object under examination and the medical object arranged in the object under examination. Further, the first planning image PI1 may be identified ID-PI and ID-SI from the multiple planning images PI, and the first monitoring image SI1 from the multiple monitoring images SI, such that the first planning image PI1 and the first monitoring image SI1 map the first physiological phase as a coincident physiological phase of the motion.

The at least one further monitoring image SIr, which is not identified ID-SI as the first monitoring image SI1 from the multiple monitoring images SI, may map at least one further physiological phase of the motion of the object under examination and the medical object arranged in the object under examination. Advantageously, in each case, positioning information POS1 and POSr about the first SI1 and the at least one further monitoring image SIr may be determined DET-POS by identification in each case of a mapping of the medical object MO in the first SI1 and the at least one further monitoring image SIr. Further, a transformation rule between the positioning information POS1 of the first monitoring image SI1 and the positioning information POSr of the at least one further monitoring image SIr may be determined DET-T. In this case, the result data set ED may, in act c), additionally be provided on the basis of the transformation rule. In particular, the result data set ED may be provided on the basis of the first planning image PI1 and adjusted positioning information POSadj. The adjusted positioning information POSadj may be provided by applying the transformation rule ADJ-POS to the positioning information POS1 and POSr.

Furthermore, the determination DET-T of the transformation rule may include a determination of a field of motion between the mappings of the medical object in the first and the at least one further monitoring image SI1 and SIr. Further, the transformation rule may be determined DET-T on the basis of the field of motion.

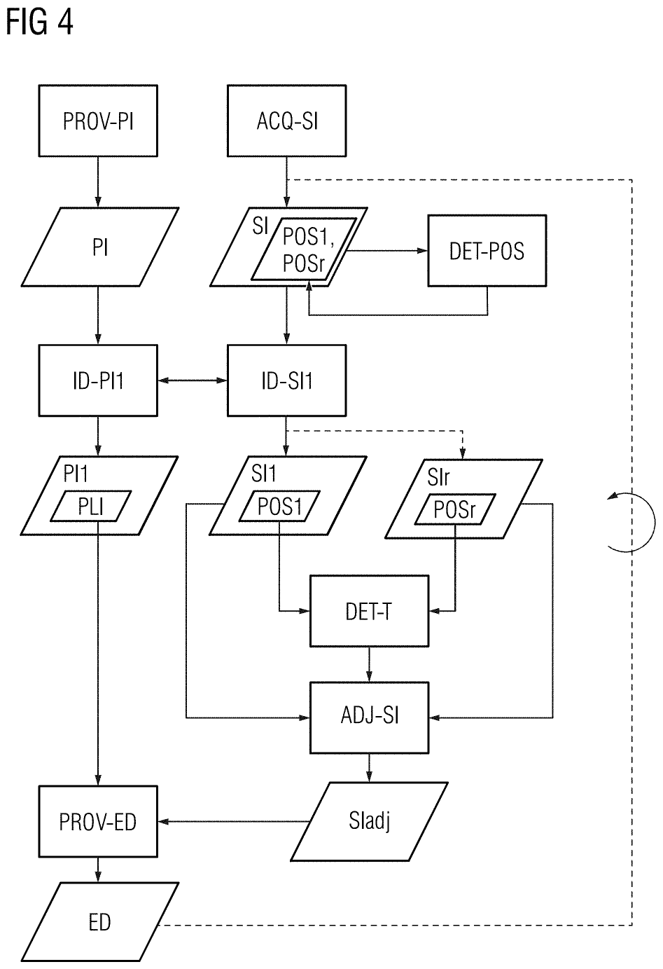

FIG. 4 shows a schematic presentation of a further advantageous embodiment of a proposed method for the provision PROV-ED of a result data set ED. In this case, in act c), the result data set ED may be provided PROV-ED on the basis of the planning information PLI and adjusted monitoring images SIadj. The adjusted monitoring images SIadj may be provided by applying the transformation rule ADJ-SI to the first SI1 and the at least one monitoring image SIr. The provision PROV-ED of the result data set ED on the basis of the planning information PLI and the adjusted monitoring images SIadj may advantageously include a summing of the adjusted monitoring images SIadj.

A motion correction for the compensation of a further motion, in particular a respiratory motion, of the object under examination may advantageously be applied to the first planning image and the first monitoring image. For this, a predefined section of the medical object may be mapped in the first planning image PI1 and the first monitoring image SI1. In this case, the motion correction may be based on the shared mapping of the predefined section in the first planning image PI1 and the first monitoring image SI1.

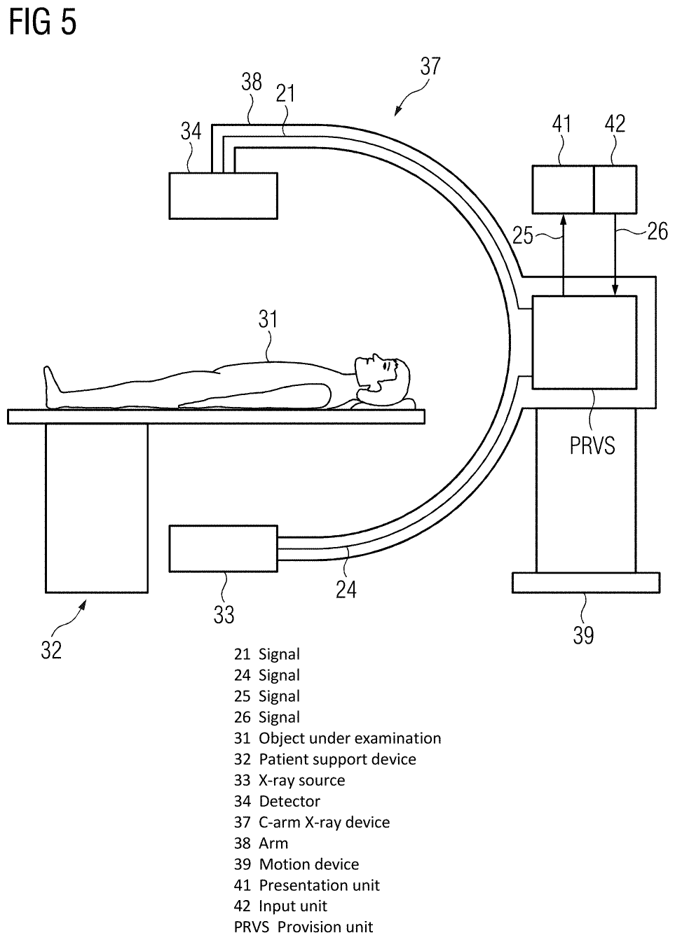

FIG. 5 schematically shows, as an example of a proposed medical imaging device, a medical C-arm X-ray device 37. The medical imaging device, in particular the medical C-arm X-ray device 37, may be configured to execute a proposed method for provision PROV-ED of a result data set ED. The medical C-arm X-ray device 37 may advantageously contain a detector 34, in particular an X-ray detector, and an X-ray source 33. For the acquisition ACQ-SI of the first monitoring image SI1, in particular the first SI1 and the at least one further monitoring image SIr, an arm 38 of the C-arm X-ray device 37 may be movably mounted about one or more axes. Further, the medical C-arm X-ray device 37 may include a motion device 39 which enables the C-arm X-ray device 37 to be moved in space.

For the acquisition ACQ-SI of the first monitoring image SI1, in particular the first SI1 and the at least one further monitoring image SIr, from the object under examination 31 arranged on a patient support device 32, a provision unit PRVS may send a signal 24 to the X-ray source 33. The X-ray source 33 may then emit an X-ray beam. When the X-ray beam, after interaction with the object under examination 31, hits a surface of the detector 34, the detector 34 may send a signal 21 to the provision unit PRVS. The provision unit PRVS may, using the signal 21, receive the first monitoring image SI1, in particular the first SI1 and the at least one further monitoring image SIr.

The medical imaging device, in particular, the medical C-arm X-ray device 37, may further be configured to acquire the first planning image PI1, in particular analogously to the first monitoring image SI1. Alternatively, the provision unit PRVS may be configured to receive the first planning image PI1.

Furthermore, the medical imaging device, in particular the medical C-arm X-ray device 37, may contain an input unit 42, for example, a keyboard, and a presentation unit 41, for example, a monitor and/or a display and/or a projector. The input unit 42 may be integrated into the presentation unit 41, for example, in the case of a capacitive and/or resistive input display. The input unit 42 may advantageously be configured to capture a user input. For this, the input unit 42 may send a signal 26 to the provision unit PRVS.

The presentation unit 41 may advantageously be configured to display a graphical presentation of the result data set ED. For this, the provision unit PRVS may send a signal 25 to the presentation unit 41.

The schematic presentations contained in the figures described are in no way to scale or in proportion.

In conclusion, it is once again noted that the methods and devices described in detail above relate solely to exemplary embodiments that may be modified by the person skilled in the art in a variety of ways, without departing from the scope of the disclosure. Further, the use of the indefinite article “a” or “an” does not rule out that the features in question may also be present multiple times. Likewise, the terms “unit” and “element” do not rule out that the components in question include multiple interacting subcomponents that if appropriate may also be distributed spatially.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the disclosure has been illustrated and described in detail with the help of the embodiments, the disclosure is not limited to the disclosed examples. Other variations may be deduced by those skilled in the art without leaving the scope of protection of the claimed disclosure.

1. A method for provision of a result data set, the method comprising:
providing a first planning image that maps a first physiological phase of a motion of an object under examination, wherein the first planning image contains planning information about a planned positioning of a medical object in the object under examination;
acquiring a first monitoring image that maps the first physiological phase of the motion of the object under examination and the medical object arranged in the object under examination; and
providing the result data set based on: (a) the first planning image and positioning information determined by identification of a mapping of the medical object in the first monitoring image, or (b) the planning information and the first monitoring image.

2. The method of claim 1, wherein at least one further monitoring image is acquired,
wherein the at least one further monitoring image maps at least one further physiological phase of the motion of the object under examination and the medical object arranged in the object under examination,
wherein, in each case, positioning information about the first monitoring image and the at least one further monitoring image is determined by identification in each case of a mapping of the medical object in the first monitoring image and the at least one further monitoring image,
wherein a transformation rule between the positioning information of the first monitoring image and the positioning information of the at least one further monitoring image is determined, and
wherein the result data set is further provided based on the transformation rule.

3. The method of claim 2, wherein the result data set is provided based on the first planning image and adjusted positioning information, and
wherein the adjusted positioning information is provided by applying the transformation rule to the positioning information.

4. The method of claim 2, wherein the result data set is provided based on the planning information and adjusted monitoring images (SIadj), and
wherein the adjusted monitoring images are provided by applying the transformation rule to the first monitoring image and the at least one further monitoring image.

5. The method of claim 4, wherein the providing of the result data set comprises a summing of the adjusted monitoring images.

6. The method of claim 2, wherein the determining of the transformation rule comprises a determination of a field of motion between mappings of the medical object in the first monitoring image and the at least one further monitoring image, and
wherein the transformation rule is determined based on the field of motion.

7. The method of claim 1, wherein the first monitoring image is acquired by a physiological signal that maps physiological phases of the motion.

8. The method of claim 1, wherein the motion of the object under examination comprises a cardiac motion, and
wherein the first physiological phase is specified as a diastolic phase of the cardiac motion.

9. The method of claim 1, wherein the medical object contains at least one marker structure, and
wherein the determining of the positioning information comprises identification of a mapping of the at least one marker structure.

10. The method of claim 9, wherein the medical object contains a spatial arrangement of multiple marker structures,
wherein the planning information contains a planning positioning in the object under examination for the spatial arrangement of the multiple marker structures, and
wherein the determining of the positioning information comprises identification of a mapping of the spatial arrangement of the multiple marker structures.

11. The method of claim 1, wherein the acquiring of the first monitoring image takes place with an acquisition geometry that corresponds to an acquisition geometry of the first planning image in respect of the object under examination.

12. The method of claim 1, wherein the first planning image maps a contrasted hollow organ of the object under examination, and
wherein, during the acquiring of the first monitoring image, the medical object is arranged at least partially in the contrasted hollow organ.

13. The method of claim 1, wherein the acquiring of the first monitoring image and the providing of the result data set are executed repeatedly.

14. The method of claim 1, wherein the first planning image and the first monitoring image are acquired by a same medical imaging device or by different medical imaging devices.

15. The method of claim 1, wherein a motion correction for a compensation of a further motion of the object under examination is applied to the first planning image and the first monitoring image (SI1).

16. The method of claim 15, wherein the further motion of the object under examination comprises a respiratory motion of the object under examination.

17. The method of claim 15, wherein a predefined section of the medical object is mapped in the first planning image and the first monitoring image, and
wherein the motion correction is based on the shared mapping of the predefined section in the first planning image and the first monitoring image.

18. The method of claim 1, wherein multiple planning images are provided that map multiple physiological phases of the motion of the object under examination,
wherein multiple monitoring images are acquired that map at least one part of the multiple physiological phases of the motion of the object under examination and the medical object arranged in the object under examination, and
wherein the first planning image is identified from the multiple planning images, and the first monitoring image from the multiple monitoring images, such that the first planning image and the first monitoring image map the first physiological phase as a coincident physiological phase of the motion.

19. A medical imaging device comprising:
an imaging unit configured to:
acquire a first planning image that maps a first physiological phase of a motion of an object under examination, wherein the first planning image contains planning information about a planned positioning of a medical object in the object under examination; and
acquire a first monitoring image that maps the first physiological phase of the motion of the object under examination and the medical object arranged in the object under examination; and
a provision unit configured to provide a result data set based on: (a) the first planning image and positioning information determined by identification of a mapping of the medical object in the first monitoring image, or (b) the planning information and the first monitoring image.

20. A non-transitory computer program product with a computer program, which is configured to be loaded into a memory of a provision unit of a medical imaging device, wherein the computer program, when executed by the provision unit, is configured to cause the medical imaging device to:
provide a first planning image that maps a first physiological phase of a motion of an object under examination, wherein the first planning image contains planning information about a planned positioning of a medical object in the object under examination;
acquire a first monitoring image that maps the first physiological phase of the motion of the object under examination and the medical object arranged in the object under examination; and
provide a result data set based on: (a) the first planning image and positioning information determined by identification of a mapping of the medical object in the first monitoring image, or (b) the planning information and the first monitoring image.
